(19) United States
(12) Patent Application Publication
(10) Pub. No.: US 2019/0380692 A1
(43) Pub. Date: Dec. 19, 2019

(54) A SUTURE ANCHOR FOR SOFT TISSUE REPAIR AND A METHOD AND A DRIVER FOR THE INSERTION THEREOF

(71) Applicant: SIGNATURE ORTHOPAEDICS EUROPE LTD, Dublin (IE)

(72) Inventors: Declan BRAZIL, Lane Cove West (AU); Shane BURWOOD, Lane Cove West (AU); Richard DALLANA, Lane Cove West (AU)

(21) Appl. No.: 15/739,309

(22) PCT Filed: Jun. 21, 2016
(86) PCT No.: PCT/AU2016/050523

§ 371 Date: Dec. 22, 2017

(30) Foreign Application Priority Data
Jun. 23, 2015 (AU) 2015902420

(51) Int. Cl. A61B 17/04

(52) U.S. Cl. CPC A61B 17/0401; A61B 2017/0464; A61B 2017/0409; A61B 2017/0441; A61B 2017/0445; A61B 2017/0456

(57) ABSTRACT

There is provided a suture anchor for soft tissue repair. The anchor is substantially elongate so as to be adapted for inserting into a blind hole or socket created in bone in use. The anchor defines externally and outwardly operative engaging means adapted for engaging the inner surface of the blind hole or socket for securing the anchor within the blind hole or socket in use. The anchor defines a distally located suture engagement adapted for engaging a suture in use, wherein, in use: the anchor is adapted for insertion into the blind hole or socket, such that: the engaging means is adapted for securing the anchor within the blind hole or socket; the engaging means is adapted for frictionally engaging the suture between the outer surface of the engaging means and the inner surface of the blind hole or socket; and the suture engagement is adapted for engaging the suture so as to substantially prevent the inadvertent disengagement of the suture from the blind hole or socket caused by the sliding of the suture between the outer surface of the anchor and the inner surface of the socket.

### FIELD OF THE INVENTION

The present invention relates to a suture anchor and in particular, but not necessarily entirely, to a suture anchor for soft tissue repair and a method and a driver for the insertion thereof comprising a suture engagement adapted for substantially preventing the inadvertent dislodgement of a suture in use.

### BACKGROUND OF THE INVENTION

Shown in FIGS. 1-3 are screw anchors of the prior art.

Specifically, FIGS. 1-2 show a screw anchor as is disclosed in US patent publication U.S. Pat. No. 6,544,281 B2 (referred to herein hereafter as D1) disclosing a method for securing soft tissue to bone wherein a blind hole or socket is created in the bone at the location the graft is to be secured.

According to D1, a suture is then passed through the graft at desired points wherein a cannulated driver is pre-loaded with a cannulated plug or screw slidably disposed onto the distal portion of the driver. A separate piece of suture is then passed through the cannula of the driver with a loop end of that suture exposed at the distal end of the driver. The ends of the suture attached to the graft are fed through the suture loop at the end of the driver. Alternatively, the graft itself may be fed through the suture loop, in which case it is not necessary to attach suture through the graft.

In another embodiment of D1, the suture loop exposed at the distal end of the cannula of the driver may be omitted, and the sutures attached to the graft may then be fed through the driver cannula from the distal end to position the graft relative to the driver. The driver is inserted into the hole with the screw or plug just outside the hole. Tension is then placed on the suture. Once adequate tension is achieved on the suture, the driver is pressed into the hole, which engages the first thread or bump of the screw or plug on the bone. The screw or plug is then fully advanced into the hole using the driver. When the screw or plug is fully inserted, the suture loop is freed and the driver is removed. The loose ends of the sutures protruding from the anchor site can be cleaned up by clipping them short.

However, a disadvantage of the screw anchor of D1 is that the suture may be inadvertently disengaged by the suture sliding between the outer surface of the screw and the inner surface of the blind hole or socket. Such a problem may be compounded for incorrectly inserted sutures comprising slack such that the suture forms a loop being susceptible to displacement during the insertion of the anchor such that the suture does not engage the head of the anchor, rather lying adjacent the head against the lateral surface of the anchor. In this manner, tension applied to the suture may cause the suture to disengage by sliding between the lateral surface of the anchor and the interior if the blind hole.

In an attempt to address such problem, US patent publication U.S. Pat. No. 8,430,909 B2 (referred to herein hereafter as D2) discloses an improved suture anchor as substantially shown in FIG. 3.

Specifically, D2 recites a pointed tip implant 451 with an eyelet or aperture for receiving the suture therethrough. The pointed tip implant 451 is advanced into the blind hole or socket prior to the insertion of the anchor 420 such that the subsequent insertion of the anchor 420 causes the distal end of the anchor to engage with the pointed tip implant 451.

However, a disadvantage of D2 is that the multiple componentry required is cumbersome requiring the surgeon to firstly thread the suture through the pointed tip implant 451 (requiring two hands, and negating the ability to use a driver suture engagement of a driver tool), then fasten the tip implant 451 to the distal end of the driver (again requiring two hands), then insert the tip implant 451 within the blind hole or socket (which may cause the tip implant 451 to become inadvertently disengage from the distal end of the inner shaft 12) and any subsequent insertion of the anchor 420 (which may not mate with the tip implant 451 should the tip implant 451 be incorrectly aligned).

The present invention seeks to provide a suture anchor, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided a suture anchor for soft tissue repair, wherein the anchor is substantially elongate so as to be adapted for inserting into a blind hole or socket created in bone in use; the anchor defines externally and outwardly operative engaging means adapted for engaging the inner surface of the blind hole or socket for securing the anchor within the blind hole or socket in use; and the anchor defines a distally located suture engagement adapted for engaging a suture in use, wherein, in use the anchor is adapted for insertion into the blind hole or socket, such that the engaging means is adapted for securing the anchor within the blind hole or socket; the engaging means is adapted for frictionally engaging the suture between the outer surface of the engaging means and the inner surface of the blind hole or socket; and the suture engagement is adapted for engaging the suture so as to substantially prevent the inadvertent disengagement of the suture from the blind hole or socket caused by the sliding of the suture between the outer surface of the anchor and the inner surface of the socket.

The engaging means may comprise a continuous screw thread.

The screw thread tapers towards the distal end of the anchor.

The screw thread may be rounded so as to substantially prevent abrasion of the suture in use.

The engagement means may be adapted for an interference fit with the inner surface of the blind hole or socket.

The suture engagement may be adapted to engage at a portion of the suture substantially perpendicular to an elongate axis of the anchor.

The suture engagement may be bifurcated so as to define a suture engaging channel therebetween.

The suture engagement may comprise opposing lobes.

The lobes are rounded at each respective distal end.

The lobes are shaped to maintain a tapered outer surface profile of the anchor.

The suture engaging channel may be spiralled along the lengthwise axis of the anchor so as to substantially trap the suture within the suture engagement.

The engaging means may comprise a continuous screw thread rotating in the direction and wherein the channel may be spiralled in the same direction.

The anchor may be substantially cannulated.

There is also provided a method for inserting the suture anchor using a driver, the driver comprising: an inner shaft having a distal driver suture engagement; an outer shaft having the inner shaft therein, the inner shaft being able to slide within the outer shaft, the distal end of the outer shaft adapted for mating with the anchor, the method comprising: providing a blind hole or socket; inserting the inner shaft through the anchor; engaging a suture using the distal driver suture engagement, inserting the distal suture engagement into the blind hole or socket; driving the outer shaft to drive the anchor into the blind hole or socket such that the suture engagement of the anchor engages the suture, and removing the inner shaft from within the anchor.

The inner shaft may further be able to rotate within the outer shaft and wherein the engaging means may comprise a screw thread and wherein driving the outer shaft may comprise rotating the outer shaft to screw the anchor into the blind hole or socket.

The suture engagement may be bifurcated so as to define a suture engaging channel therebetween and wherein the suture engaging channel may be spiralled along the elongate axis of the anchor such that, when screwing the anchor into the blind hole or socket, these spiralled suture engaging channel may rotate about the suture so as to entrap the suture therein.

The depth of the suture engaging channel may conform substantially with an extent of a driver suture engagement.

According to another aspect, there is provided a driver for inserting the suture anchor, the driver comprising: an inner shaft having a distal suture engagement, the inner shaft adapted for insertion through the suture anchor; and an outer shaft having the inner shaft therein, the inner shaft being able to slide within the outer shaft such that the distal end of the outer shaft is able to drive the anchor along the elongate axis of the anchor while the inner shaft extends through the suture anchor.

The inner shaft may further be adapted for rotation within the outer shaft and wherein the distal end of the outer shaft may be configured for mating with the suture anchor so as to be able to screw the suture anchor.

Other aspects of the invention are also disclosed.

### BRIEF DESCRIPTION OF THE DRAWINGS

Notwithstanding any other forms which may fall within the scope of the present invention, a preferred embodiments of the disclosure will now be described, by way of example only, with reference to the accompanying drawings in which:

FIGS. 1-3 shows screw anchors of the prior art;

FIG. 4 shows a screw anchor in accordance with embodiments of the present disclosure;

FIGS. 5-13 show a method of inserting the screw anchor of FIG. 4 in accordance with embodiments of the present disclosure; and

FIG. 14 shows a suture engaged by a suture engagement of the suture anchor of FIG. 1 in accordance with embodiments of the present disclosure.

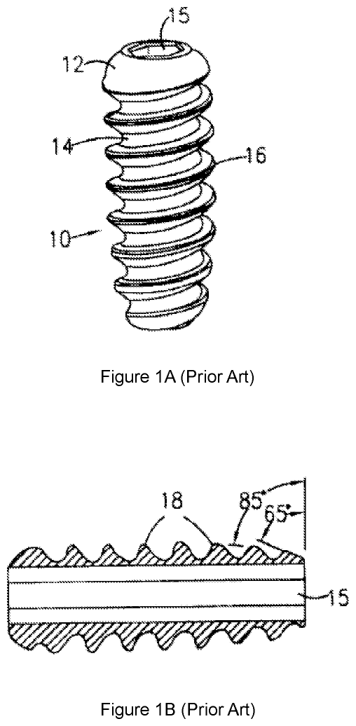

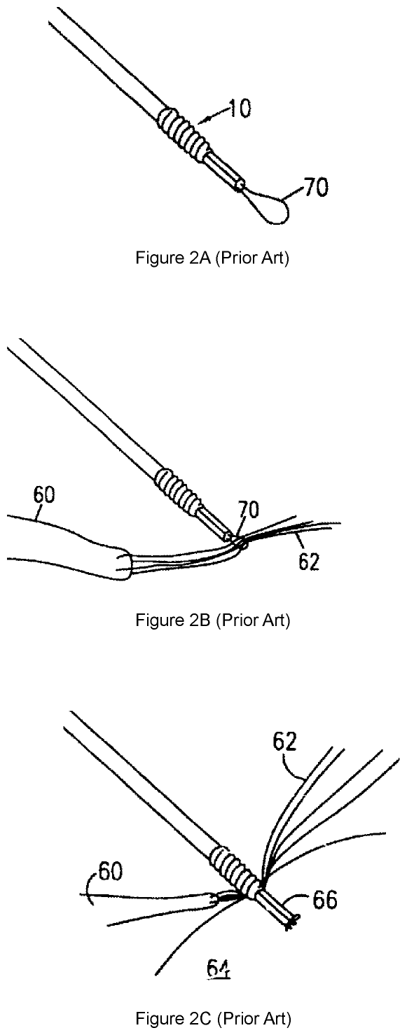

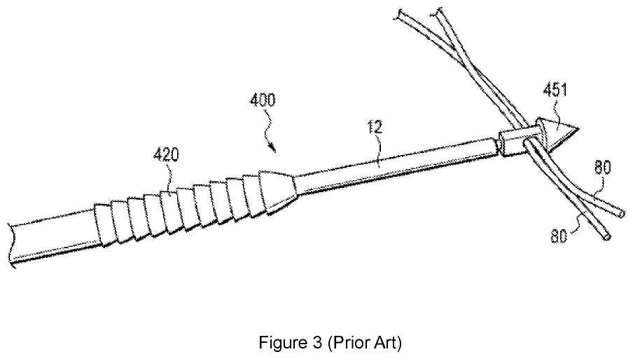

### DESCRIPTION OF EMBODIMENTS

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure.

Before the structures, systems and associated methods relating to the suture anchor are disclosed and described, it is to be understood that this disclosure is not limited to the particular configurations, process steps, and materials disclosed herein as such may vary somewhat. It is also to be understood that the terminology employed herein is used for the purpose of describing particular embodiments only and is not intended to be limiting since the scope of the disclosure will be limited only by the claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterised by,” and grammatical equivalents thereof are inclusive or open-ended terms that do not exclude additional, unrecited elements or method steps.

It should be noted in the following description that like or the same reference numerals in different embodiments denote the same or similar features.

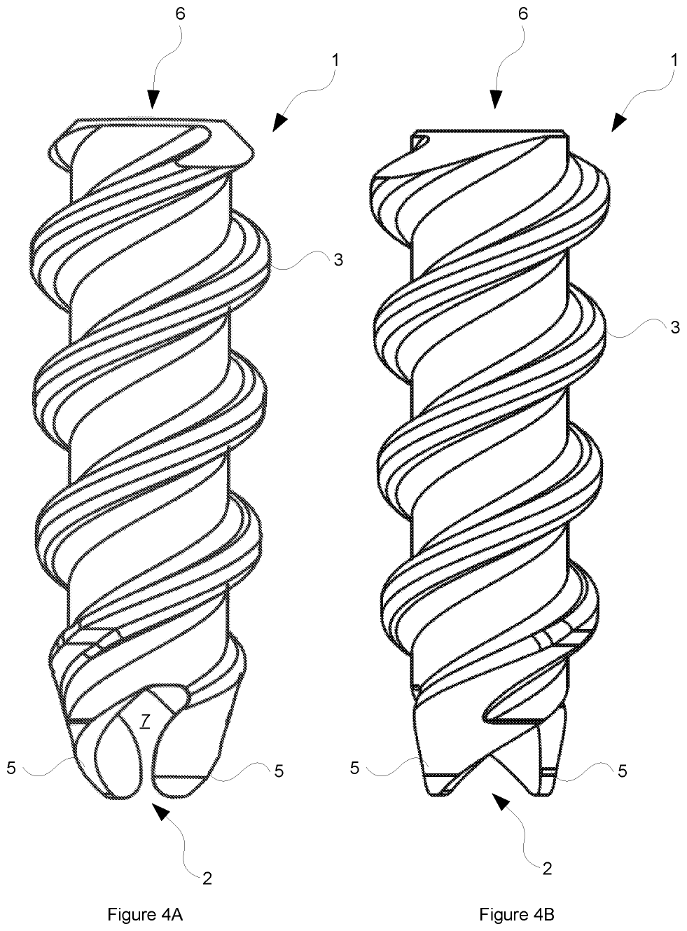

Turning now to FIG. 4, there is shown a suture anchor 1 which may be used for applications such as soft tissue repair.

FIG. 4B shows the suture anchor 1 rotated 90° around the elongate axis of the anchor 1 when compared to the same suture anchor 1 as substantially shown in FIG. 4A.

Specifically, the anchor 1 is substantially elongate so as to be adapted for inserting into a blind hole or socket created in bone in use. Furthermore, the anchor 1 defines externally and outwardly operative engaging means 3 adapted for engaging the inner surface of the blind hole or socket for securing the anchor 1 within the blind hole or socket in use.

Furthermore, as can be seen, the anchor 1 defines a distantly located suture engagement 2 adapted for engaging a suture in use.

As such, in use, the anchor 1 is adapted for insertion into the blind hole or socket such that the engaging means 3 is adapted for securing the anchor 1 within the blind hole or socket. Furthermore, the engaging means 3 is adapted for frictionally engaging the suture between the outer surface of the engaging means 3 and the inner surface of the blind hole or socket.

Furthermore, the suture engagement 2 is adapted for engaging the suture so as to substantially prevent the inadvertent disengagement of the suture from the blind hole or socket caused by the sliding of the suture between the outer surface of the anchor 1 and the inner surface of the socket.

In a preferred embodiment, the engaging means 3 comprises a continuous screw thread as substantially shown in FIG. 4 so as to allow the anchor 1 to be screwed into the blind hole or socket. As can also be seen, the screw thread may taper towards the distal end of the anchor 1.

In a preferred embodiment, the screw thread 3 is rounded so as to substantially prevent abrasion of the suture in use.

It should be noted that in other embodiments, other engaging means 3 may be utilised within the purposive scope of engaging the anchor 1 within the socket and frictionally engaging the suture including non-spiralled configurations such as ribbing, arrowhead bosses and the like.

Now, the suture engagement 2 is adapted for engaging a portion of the suture across the distal end of the anchor 1, being substantially perpendicular to an elongate axis of the anchor 1.

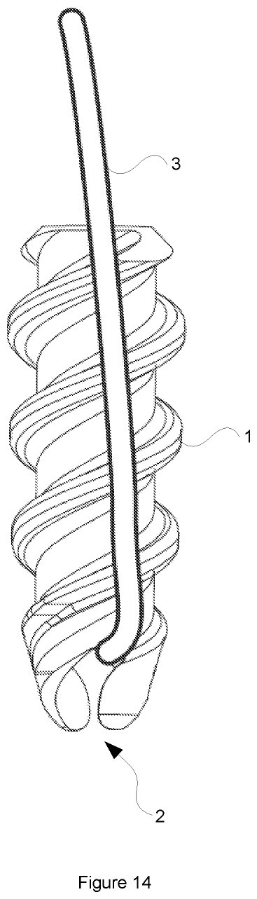

Specifically, as can best be seen from FIG. 14, the suture 3 travels downwards (substantially perpendicular to the elongate axis of the anchor 1) towards the suture engagement 2 so as to then traverse the suture engagement 2 substantially perpendicular to the elongate axis of the anchor 1 and thereafter transition upwardly again so as to travel upwards out of the blind hole or socket substantially perpendicular to the elongate axis of the anchor 1.

Returning again to FIG. 4, as can be seen, the suture engagement 2 is preferably bifurcated so as to define a suture engaging channel 7 therebetween.

As can be appreciated, the suture engaging channel 7 may comprise sufficient depth so as to provide the purposive advantage of engaging the suture 3 so as to substantially prevent the suture 3 from escaping from the blind hole or socket by sliding between the outer surface of the anchor 1 in the inner surface of the blind hole or socket, especially for incorrectly inserted sutures.

Further preferably, the suture engagement 2 comprises a pair opposing lobes 5. In a preferred embodiment, the lobes 2 are flattish projections extending from the distal end of the anchor 1 each and having rounded outer lateral surfaces confirming to the substantially cylindrical shape of the body of the anchor 1. Furthermore, the lobes 5 may be shaped to maintain a tapered outer surface profile of the anchor 1.

In the embodiment shown, the lobes 5 are rounded at each respective distal end.

Now, in a preferred embodiment, the suture engaging channel 7 is spiralled along the lengthwise axis of the anchor 1 so as to substantially entrap the suture 3 therein.

Specifically, the perpendicular portion of the suture 3 extending across the distal end of the anchor 1 within the channel 7 perpendicular the elongate of the axis 1 is unlikely to be able to rotate so as to conform with the spiralling of the channel 7 along the elongate axis of the anchor 1 to be disengaged therefrom, especially on account of being placed under tension from the adjacent parallel portions of the suture 3 extending along the elongate axis of the anchor 1 or the fictional engagement by the engaging means on the adjacent parallel portions of the suture 3 preventing the provision of slack to the perpendicular portion of the suture 3.

As can be further seen from the embodiment provided in FIG. 4, the continuous screw thread 3 is adapted to rotate in a direction and wherein the spiralled channel 7 similarly rotates in the same direction as that of the screw thread 3. As such, during the insertion of the anchor 1, the spiralled channel 7 is naturally adapted to engage and trap the suture 3 as the anchor 1 is screwed into the blind hole or socket.

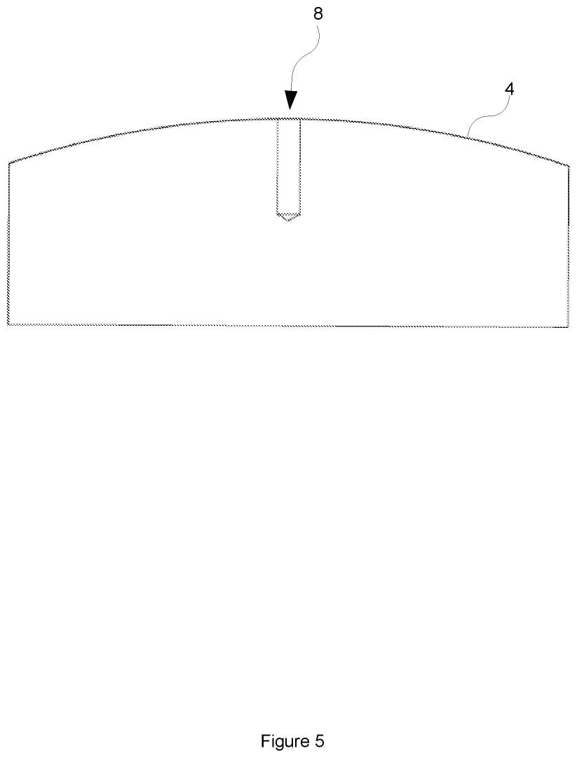

With reference now to FIGS. 5-13, there will be described a method for the anchoring of the suture 3 utilising the anchor 1.

Specifically, referring to FIG. 5 a blind hole or socket 8 is created within the bone 4.

In a preferred embodiment, the anchor 1 is adapted for utilisation with a driver. Specifically, in a preferred embodiment, the driver comprises an inner shaft comprising a suture engagement (such as a distantly located claw, eyelet or the like) and an outer shaft through which the inner shaft slidably travels, the outer shaft comprising an anchor engagement 1 (such as a screwdriver engagement) adapted to allow the screwing of the anchor 1 into the blind hole or socket in use.

As such, in a preferred embodiment, the anchor 1 is substantially cannulated. In this manner, the anchor 1 is adapted for receiving the inner shaft of the driver therethrough. Furthermore, the anchor 1 comprises a bore 6 comprising a screwdriver engagement portion adapted for engaging the outer shaft of the driver. In one embodiment, the screwdriver engaging portion may be substantially hexagonal.

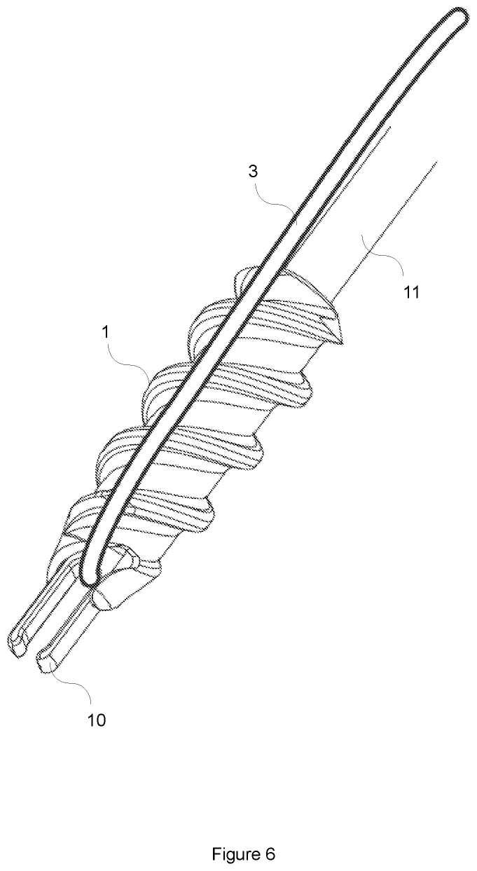

Specifically, referring to FIG. 6, the suture 3 is engaged by the driver suture engagement 10 of the inner shaft 11, being a claw-type arrangement in the embodiment shown. It should be noted that whereas FIG. 6 shows the anchor 1 located adjacent the distal end of the shaft 11, in embodiments, the anchor 1 may be located away from the distal end on account of being able to slide along the shaft 11.

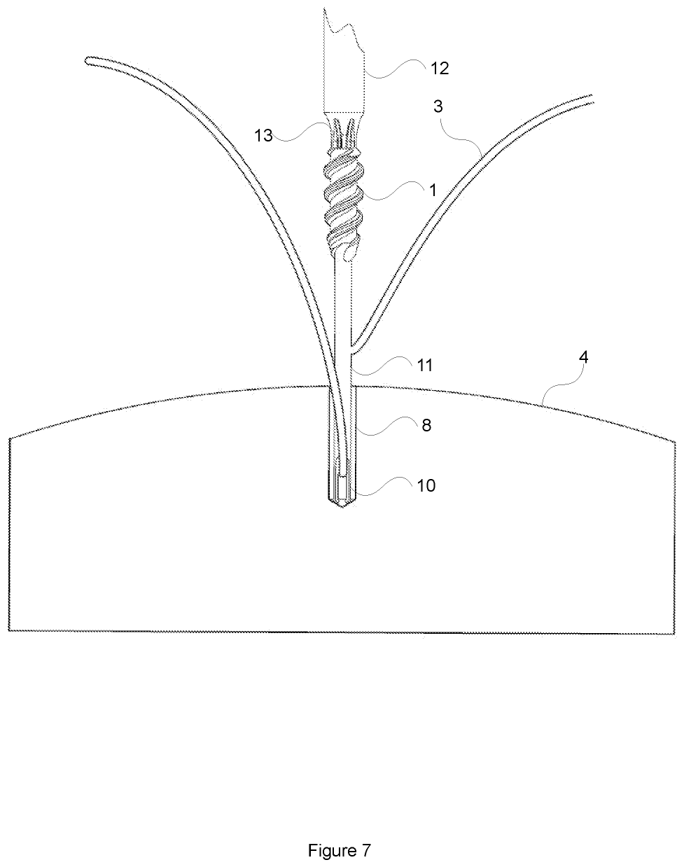

Referring now to FIG. 7, the distally located driver suture engagement 10 of the inner shaft 11 is inserted within the socket 8 so as to insert the suture 3 at least partially within the socket 8.

As shown in FIG. 7, the driver comprises the outer shaft 12 within which the inner shaft 11 is able to slide and rotate. Furthermore, the outer shaft 12 comprises a screwdriver engagement 13 adapted for mating with the inwardly located screwdriver engagement portion of the bore 6 of the anchor 1. In this manner, the screwdriver engagement 13 is adapted for screwing the anchor 1 into the socket 8 in the manner described herein.

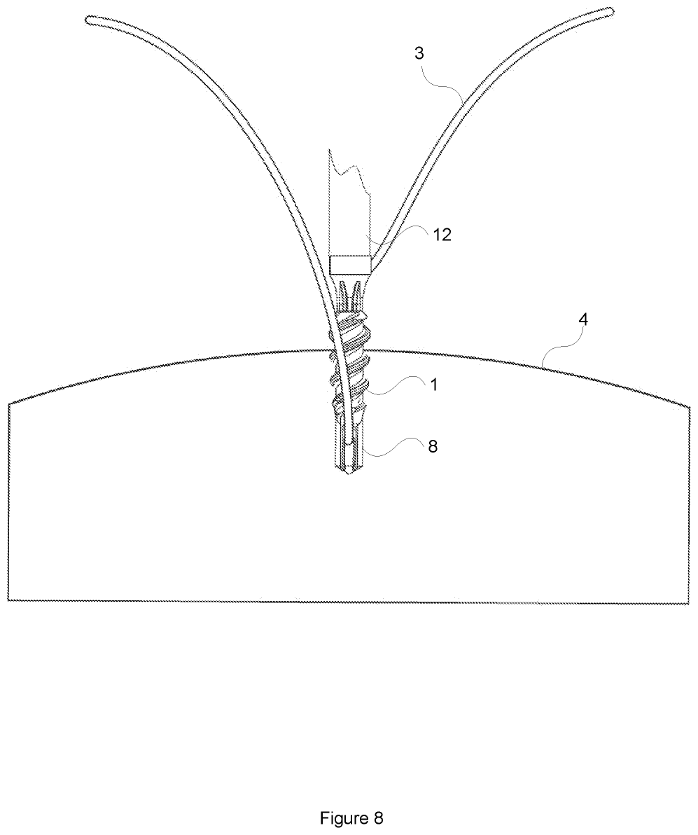

Referring now to FIG. 8, there is shown the screwing of the anchor 1 within the socket 8. Specifically, the outer shaft 12 is rotated and driven towards the socket 8 so as to screw the anchor 1 within the socket 8.

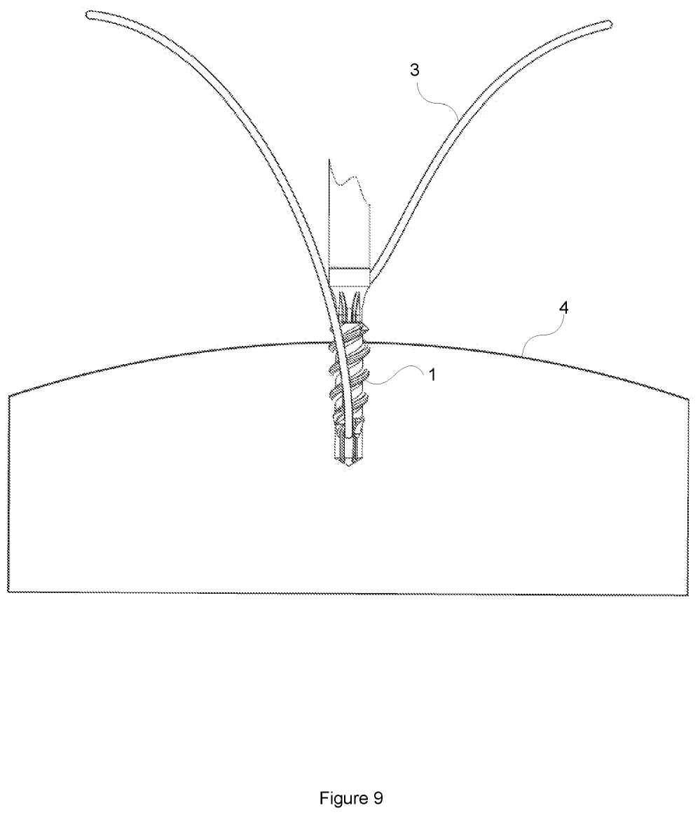

Referring to FIG. 9, at a certain depth, the suture engagement 2 of the anchor 1 engages the suture held in place by the driver suture engagement 10.

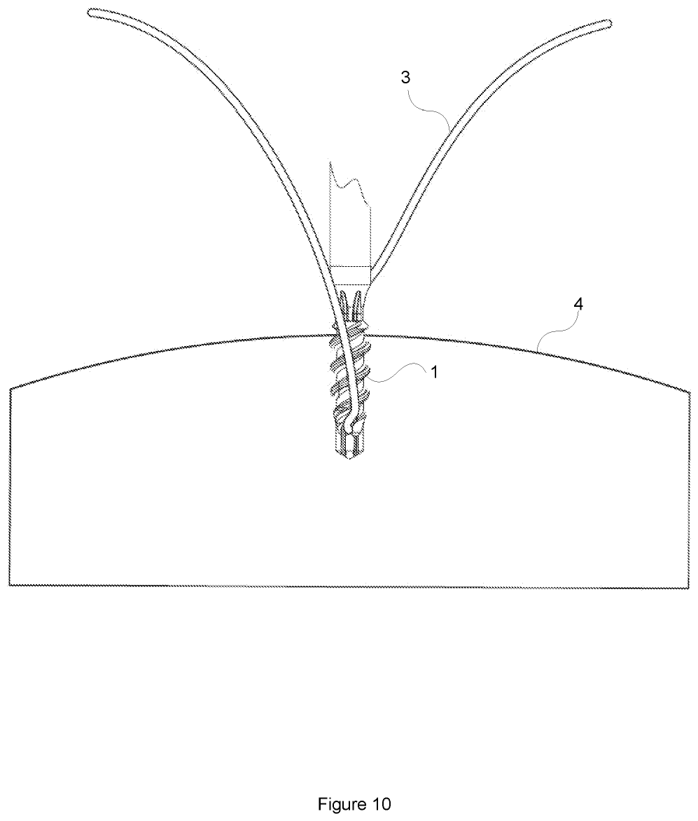

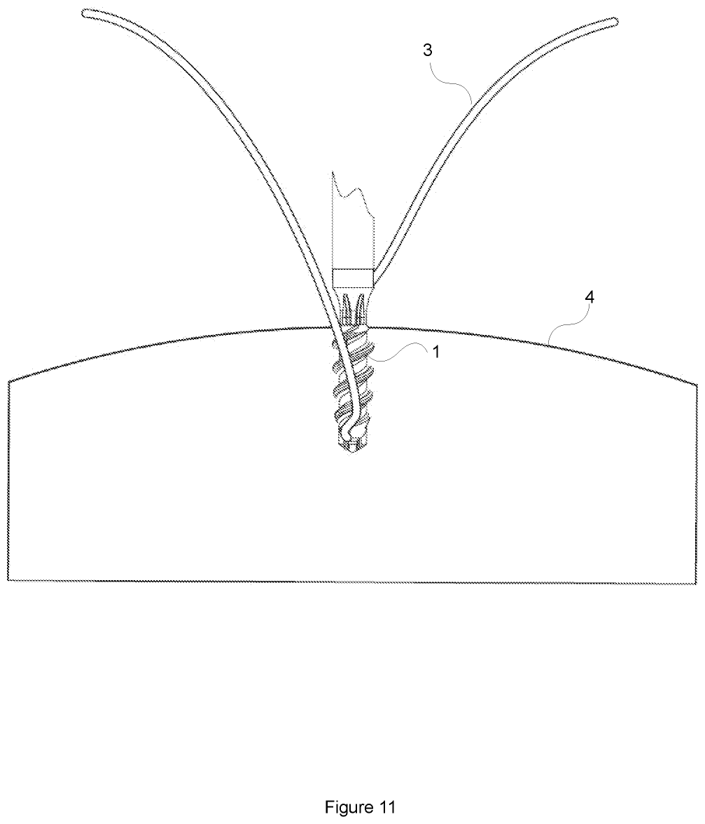

FIG. 10 shows the initial engagement of the suture 3 by the suture engagement 2 as the anchor 1 is screwed into the socket 8 and FIG. 11 shows the further engagement of the suture 3 by the suture engagement 2.

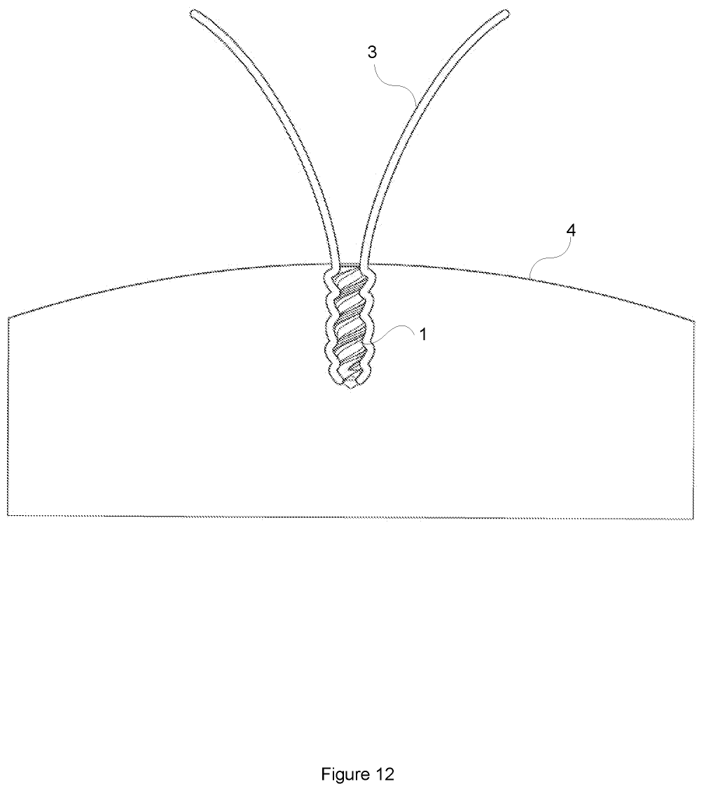

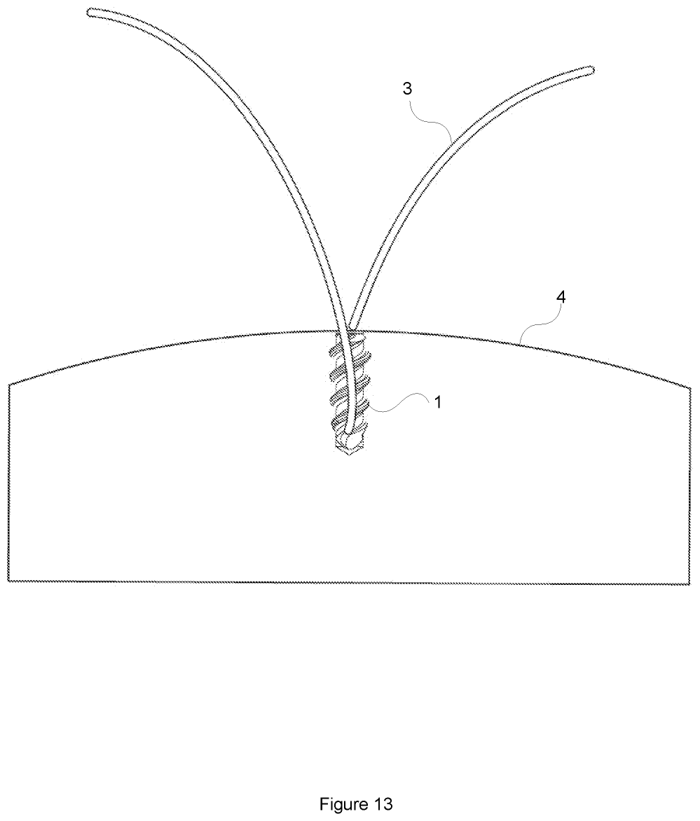

Once the suture 3 has been engaged by the anchor 1 in this manner, the inner shaft 1 of the driver is removed so as to leave the suture 3 in place. Specifically, referring to FIG. 12, there is shown a side view showing the engagement of the suture 3 by the anchor 1. Furthermore, FIG. 13 shows the view rotated 90° with respect to that shown in FIG. 12 showing the engagement of the suture 3 by the anchor 1.

It should be noted that, in one embodiment, the depth of the suture engaging channel 7 may substantially conform to the extent of the driver suture engagement 10 of the inner shaft 11. In this manner, when the suture engagement 2 engages the suture located at the apex of the driver suture engagement 10, the anchor 1 may be screwed such that the suture 3 does not become unduly twisted about the suture engagement 2 which may induce undue stress on the suture 3.

### Interpretation

### Embodiments:

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

1. A suture anchor for soft tissue repair, wherein:
the anchor is substantially elongate so as to be adapted for inserting into a blind hole or socket created in bone in use;
the anchor defines externally and outwardly operative engaging means adapted for engaging the inner surface of the blind hole or socket for securing the anchor within the blind hole or socket in use; and
the anchor defines a distally located suture engagement adapted for engaging a suture in use, wherein, in use:
the anchor is adapted for insertion into the blind hole or socket, such that:
the engaging means is adapted for securing the anchor within the blind hole or socket;
the engaging means is adapted for frictionally engaging the suture between the outer surface of the engaging means and the inner surface of the blind hole or socket; and
the suture engagement is adapted for engaging the suture so as to substantially prevent the inadvertent disengagement of the suture from the blind hole or socket caused by the sliding of the suture between the outer surface of the anchor and the inner surface of the socket.

2. A suture anchor as claimed in claim 1, wherein the engaging means comprises a continuous screw thread.

3. A suture anchor as claimed in claim 2, wherein the screw thread tapers towards the distal end of the anchor.

4. A suture anchor as claimed in claim 2, wherein the screw thread is rounded so as to substantially prevent abrasion of the suture in use.

5. A suture anchor as claimed in claim 1, wherein the engagement means is adapted for an interference fit with the inner surface of the blind hole or socket.

6. A suture anchor as claimed in claim 1, wherein the suture engagement is adapted to engage at a portion of the suture substantially perpendicular to an elongate axis of the anchor.

7. A suture anchor as claimed in claim 6, wherein the suture engagement is bifurcated so as to define a suture engaging channel therebetween.

8. A suture anchor as claimed in claim 7, wherein the suture engagement comprises opposing lobes.

9. A suture anchor as claimed in claim 8, wherein the lobes are rounded at each respective distal end.

10. A suture anchor as claimed in claim 8, wherein the lobes are shaped to maintain a tapered outer surface profile of the anchor.

11. A suture anchor as claimed in claim 7, wherein the suture engaging channel is spiralled along the lengthwise axis of the anchor.

12. A suture anchor as claimed in claim 11, wherein the engaging means comprises a continuous screw thread and wherein the channel is spiralled in the same direction as that of the screw thread.

13. A suture anchor as claimed in claim 1, wherein the anchor is cannulated.

14. A method for inserting a suture anchor as claimed in claim 13 using a driver, the driver comprising:
an inner shaft having a distal driver suture engagement;
an outer shaft having the inner shaft therein, the inner shaft being able to slide within the outer shaft, the distal end of the outer shaft adapted for mating with the anchor, the method comprising:
providing a blind hole or socket;
inserting the inner shaft through the anchor;
engaging a suture using the distal driver suture engagement,
inserting the distal suture engagement into the blind hole or socket;
driving the outer shaft to drive the anchor into the blind hole or socket such that the suture engagement of the anchor engages the suture,
removing the inner shaft from within the anchor.

15. A method as claimed in claim 14, wherein the inner shaft is further able to rotate within the outer shaft and wherein the engaging means comprises a screw thread and wherein driving the outer shaft comprises rotating the outer shaft to screw the anchor into the blind hole or socket.

16. A method as claimed in claim 15, wherein the suture engagement is bifurcated so as to define a suture engaging channel therebetween and wherein the suture engaging channel is spiralled along the elongate axis of the anchor such that, when screwing the anchor into the blind hole or socket, these spiralled suture engaging channel rotates about the suture so as to entrap the suture therein.

17. A method as claimed in claim 16, wherein the depth of the suture engaging channel conforms substantially with an extent of a driver suture engagement.

18. A driver for inserting a suture anchor as claimed in claim 13, the driver comprising:
an inner shaft having a distal suture engagement, the inner shaft adapted for insertion through the suture anchor;
an outer shaft having the inner shaft therein, the inner shaft being able to slide within the outer shaft such that the distal end of the outer shaft is able to drive the anchor along the elongate axis of the anchor while the inner shaft extends through the suture anchor and the distal suture engagement engages a suture.

19. A driver as claimed in claim 18, wherein the inner shaft is further adapted for rotation within the outer shaft and wherein the distal end of the outer shaft is configured for mating with the suture anchor so as to be able to screw the suture anchor.
